(19) United States
(12) Patent Application Publication
(10) Pub. No.: US 2021/0244629 A1
(43) Pub. Date: Aug. 12, 2021

(54) MULTI-LAYER DEODORANT APPLICATOR STICKS HAVING MULTIPLE SENSORY CHARACTERISTICS

(71) Applicant: Kenneth Anderson, Irvine, CA (US)

(72) Inventor: Kenneth Anderson, Irvine, CA (US)

(21) Appl. No.: 17/132,450

(22) Filed: Dec. 23, 2020

Related U.S. Application Data
(60) Provisional application No. 62/972,514, filed on Feb. 10, 2020.

(51) Int. Cl. A61K 8/02; A61Q 15/00

(52) U.S. Cl. CPC A61K 8/0237; A61Q 15/00

(57) ABSTRACT

Multi-layer deodorant applicator sticks are disclosed to be applied to the body of a user to prevent or mask body odor. Each deodorant applicator stick includes a first deodorant composition layer having a first sensory characteristic, such as a particular fragrance, and at least one second deodorant composition layer having a different sensory characteristic. The first and second deodorant composition layers are joined to one another (e.g., within a container) so that the respective first and second sensory characteristics thereof are available to the user either simultaneously or one at a time as the multi-layer deodorant applicator stick wears down over time. That is to say, a customized mixture of the first and second deodorant composition layers having the first and second sensory characteristics can be applied to the body of the user from a single deodorant applicator stick so as to avoid the necessity that the user have access to a second deodorant applicator stick.

### CROSS-REFERENCE To RELATED APPLICATIONS

This application is related to Provisional Patent Application No. 62/972,514 filed Feb. 10, 2020.

### BACKGROUND OF THE INVENTION

### 1. Field of the Invention

This invention relates primarily to deodorant applicator sticks, each of which having a multi-layer deodorant composition. Each layer of each multi-layer deodorant applicator stick has a different sensory characteristic such as, for example, fragrance so as to provide a user with a customized mixture of characteristics as the deodorant applicator stick is applied to the user's body and wears down over time.

### 2. Background Art

Conventional deodorant applicator sticks that are used to prevent or mask body odor caused by perspiration are known to have a homogeneous composition. That is, the conventional deodorant applicator stick consists of a single uniform solid or semi-solid material that provide a user with a corresponding single sensory characteristic such as color, fragrance and hardness. Therefore, should the user wish to vary a characteristic of his or her deodorant applicator stick, at least one additional deodorant applicator stick must be purchased having a different characteristic. This necessitates that the user have access to two or more different deodorant applicator sticks at the same time while incurring the increased cost associated therewith.

To overcome the disadvantages of conventional single characteristic deodorant applicator sticks it would be desirable for the user to have access to a single multi-layer deodorant applicator stick wherein each layer of the stick has its own particular sensory characteristic by which to simultaneously offer the user a variety of characteristics.

### SUMMARY OF THE INVENTION

According to a preferred embodiment, disclosed herein are deodorant applicator sticks are disclosed herein of the kind that are traditionally applied to a user's body to prevent or mask body odor caused by perspiration that is commonly encountered by active individuals. Rather than consisting of a single homogeneous material having a correspondingly single sensory characteristic such as, for example, fragrance, color or texture, each of the deodorant applicator sticks of this invention is comprised of two or more layers of deodorant composition material with each layer having a different sensory characteristic. In this manner, a user will advantageously need to have access to only a single multi-layer deodorant applicator stick wherein the multiple layers thereof have respective sensory characteristics that, in some embodiments, will vary as the deodorant applicator stick wears down over time. The multi-layer deodorant applicator sticks herein disclosed are preferably cylindrical to be housed in and dispensed from an optional cylindrical container having a platform that is lifted in order to raise the stick outwardly from the container as it is consumed during use.

In one embodiment, a multi-layer deodorant applicator stick includes two or more cylindrical layers that are concentrically aligned, such that one cylindrical layer surrounds an adjacent cylindrical layer. In another embodiment, a multi-layer deodorant applicator stick includes two or more disc-like layers that lie one on top of the other. In another embodiment, a multi-layer deodorant applicator stick includes two or more vertically extending layers that lie side-by-side one another. In another embodiment, a multi-layer deodorant applicator stick includes two or more angled layers that lie one above the other and that have irregular (i.e., dissimilar) shapes, such that an upper one of the angled layers is fully consumed prior to the consumption of an angled lower layer that extends from a location adjacent the top to a location below the bottom of the upper layer. In another embodiment, a multi-layer deodorant applicator stick includes an upstanding cylindrical first layer and a second layer that extends radially through the upstanding cylindrical first layer.

By virtue of the multi-layer deodorant applicator sticks herein disclosed, the user will experience one or a variety of different sensory characteristics of the deodorant applicator sticks as they are consumed over time without requiring that the user have access to more than a single deodorant applicator stick.

### BRIEF DESCRIPTION OF THE DRAWINGS

FIG. 1 shows a first multi-layer deodorant applicator stick including first and second layers being concentrically aligned and having different sensory characteristics associated therewith;

FIG. 2 shows the first multi-layer deodorant applicator stick having more than the first and second concentrically aligned layers shown in FIG. 1;

FIG. 3 shows a second multi-layer deodorant applicator stick including first and second disc-like layers lying one above the other and haying different sensory characteristics associated therewith;

FIG. 4 shows the second multi-layer deodorant applicator stick having more than the first and second disc-like layers shown in FIG. 3;

FIG. 5 shows a third multi-layer deodorant applicator stick including first and second vertically extending layers lying side-by-side one another and having different sensory characteristics associated therewith;

FIG. 6 shows the third multi-layer deodorant applicator stick having more than the first and second vertically extending layers shown in FIG. 5;

FIG. 7 shows a fourth multi-layer deodorant applicator stick including first and second angled and irregularly shaped layers lying one above the other and having different sensory characteristics associated therewith;

FIG. 8 shows the fourth multi-layer deodorant applicator stick having more than the first and second angled and irregularly shaped layers shown in FIG. 7;

FIG. 9 shows a fifth multi-layer deodorant applicator stick including an upstanding cylindrical first layer and a second layer extending radially through the cylindrical first layer, wherein the first and second layers have different sensory characteristics associated therewith;

FIG. 10 is a cross-section of the fifth multi-layer deodorant applicator stick taken along lines 10-10 of FIG. 9; and

FIG. 11 is an exploded view showing one example of an optional container within which any of the multi-layer deodorant applicator sticks of FIGS. 1-10 may be housed to be dispensed to a user.

### DESCRIPTION OF THE PREFERRED EMBODIMENT

Turning now to the drawings and to a preferred embodiment of this invention, multi-layer deodorant applicator sticks are shown and described which, when applied to a user's body, provide him or her with more than a single sensory characteristic as would otherwise be available from conventional deodorant applicator sticks having a homogeneous composition. This advantage will be available without the user having to purchase and use different deodorant applicator sticks one at a time having correspondingly different sensory characteristics. By way of example only, the multiple layers of the deodorant applicator sticks herein disclosed provide the user with a variety of fragrances, colors and/or textures that will be available simultaneously or one at a time. Each multi-layer deodorant applicator stick can be manufactured and used with or without a container from which the stick is dispensed. By way of example only, individual multi-layer deodorant applicator sticks can be manufactured as replacements for deodorant applicator sticks which have been consumed by the user.

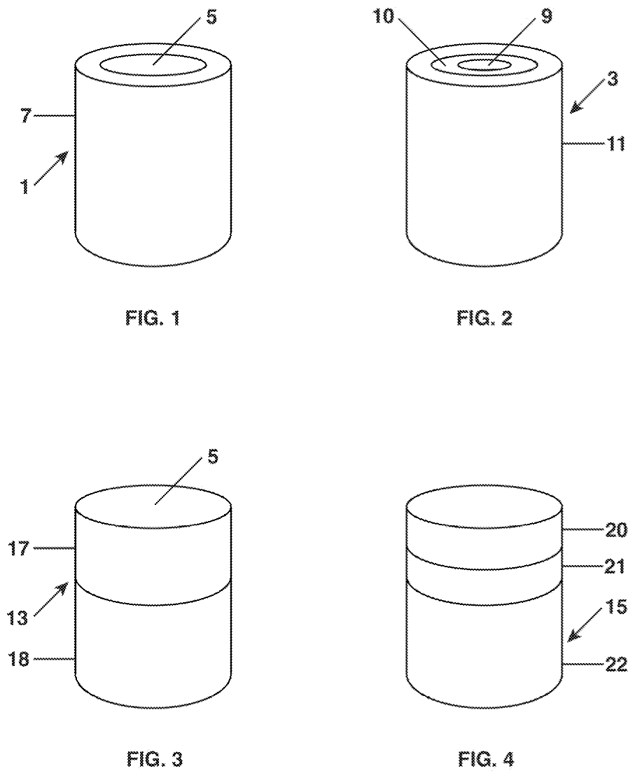

Referring initially to FIGS. 1 and 2 of the drawings, first and second multi-layer deodorant applicator sticks 1 and 3 are shown having a cylindrical configuration and respective sets of two or more deodorant composition layers that are concentrically aligned relative to one another. In this regard, although the multi-layer deodorant applicator configurations are referred to herein as “sticks,” this description should not be considered as a limitation as to the actual shape of the configuration.

In the case of FIG. 1, the multi-layer deodorant applicator stick 1 includes concentrically aligned inner and outer deodorant composition layers 5 and 7 having different sensory characteristics and being arranged so that the outer layer 7 surrounds the inner layer 5. That is, the inner layer 5 of deodorant applicator stick 1 is cylindrical, and the outer layer 7 is tubular so as to lie in surrounding engagement with the inner layer 5. In the case of FIG. 2, the multi-layer deodorant applicator stick 3 includes three concentrically aligned deodorant composition layers 9, 10 and 11 having different sensory characteristics and being arranged with the layers surrounding one another. In particular, the inner layer 9 of deodorant applicator stick 3 is cylindrical, and the other deodorant layers 10 and 11 are tubular, such that the intermediate layer 10 lies in surrounding engagement with the inner layer 9, and the outer layer 11 lies in surrounding engagement with the intermediate layer 10.

In the cases of the deodorant applicator sticks 1 and 3 of FIGS. 1 and 2, the multiple deodorant composition layers thereof are simultaneously applied to the user. In this regard, the precise number of concentrically aligned layers and the corresponding sensory characteristics offered thereby is a matter of choice and not to be considered a limitation of this invention.

Turning now to FIGS. 3 and 4 of the drawings, third and fourth multi-layer deodorant applicator sticks 13 and 15 are shown having a cylindrical configuration and respective sets of two or more deodorant composition layers that are stacked one above the other. In the case of FIG. 3, the multi-layer deodorant applicator stick 13 includes identically sized upper and lower disc-shaped deodorant layers 17 and 18 having different sensory characteristics and being arranged one above the other. That is, the upper disc-shaped deodorant layer 17 of deodorant applicator stick 13 lays on the top of and against the opposing lower disc-shaped layer 18. In the case of FIG. 4, the multi-layer deodorant applicator stick 15 includes three disc-shaped deodorant layers 20, 21 and 22 having different sizes, different sensory characteristics and being arranged one on top of the other, such that the intermediate layer 21 lays over and against the bottom layer 22 and the top layer 20 lays over and against the intermediate layer 21. In each of the cases of the deodorant applicator sticks 13 and 15 of FIGS. 3 and 4, the multiple deodorant composition layers thereof are applied to the user one at a time.

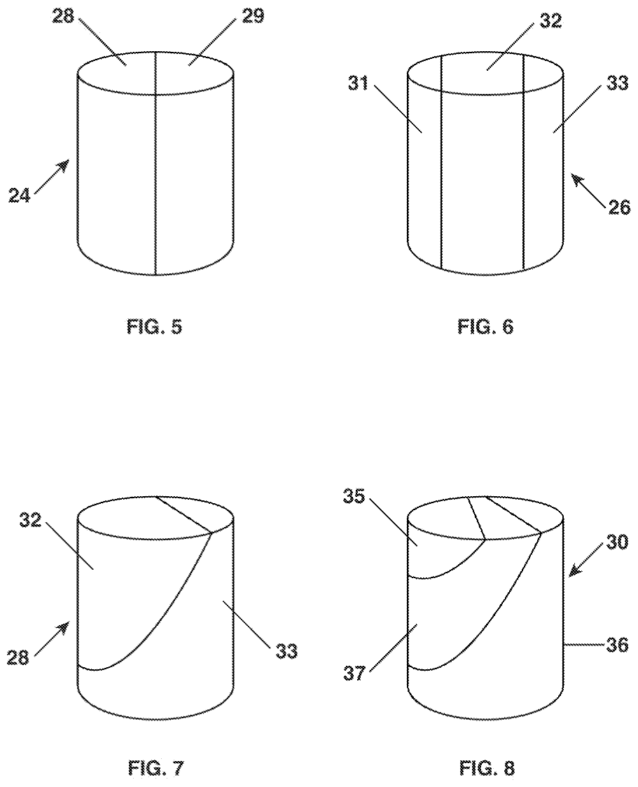

In FIGS. 5 and 6 of the drawings, fifth and sixth multi-layer deodorant applicator sticks 24 and 26 are shown having a cylindrical configuration and respective sets of two or more vertically extending deodorant composition layers. The multi-layer deodorant applicator stick 24 includes identically sized and parallel aligned left side and right side semi-cylindrical deodorant layers 28 and 29 having different sensory characteristics and being arranged side-by-side one another. That is, the vertically extending left side semi-cylindrical deodorant layer 28 of deodorant applicator stick 24 lies against the opposing vertically extending right side semi-cylindrical deodorant layer 29. In the case of FIG. 6, the multi-layer deodorant applicator stick 26 includes vertically extending and parallel aligned left side, right side and intermediate deodorant layers 31, 32 and 33 having different sizes and different characteristics and being arranged side-by-side. The vertically extending intermediate layer 32 lies between and against the vertically extending left side and right side layers 31 and 33. in each of the cases of the deodorant applicator sticks 24 and 26 of FIGS. 5 and 6, the multiple deodorant composition layers thereof are applied simultaneously to the user.

FIGS. 7 and 8 of the drawings show seventh and eighth multi-layer deodorant applicator sticks 28 and 30 having a cylindrical configuration and respective sets of angled deodorant composition layers that lie face-to-face one another and that have both sensory characteristics and irregular shapes and sizes that differ from one another. In the case of FIG. 7, the cylindrical multi-layer deodorant applicator stick 28 includes a first angled deodorant layer 32 that encompasses the upper left corner at a firs side of the deodorant applicator stick 28 and a second angled deodorant layer 33 that encompasses both the upper right corner and the lower right corner at the opposite side of the deodorant applicator stick 28. The second deodorant layer 33 also encompasses the lower left corner at the first side of stick 28 so as to extend below the first angled layer 32. Initially, the tops of the first and second angled layers 32 and 33 lie adjacent one another at the top of the cylindrical deodorant applicator stick 28 at which to be initially accessible to the user. However, the area of the first angled deodorant layer 32 at the top of the deodorant applicator stick 28 is greater than the area of the angled second deodorant layer 33 that lies adjacent the top of the first layer 32 at the top of the deodorant applicator stick 28. In this same regard, the volume of the second layer 33 is greater than the volume of the first layer 32.

In the case of FIG. 8, the cylindrical multi-layer deodorant applicator stick 30 includes an angled first deodorant layer 35 that encompasses the upper left corner of the deodorant applicator stick 30 at a first side thereof, an angled second deodorant layer 36 that encompasses the upper and lower right corners at the opposite side of the deodorant applicator stick 30 as well as the lower left corner at the first side of the deodorant applicator stick 30, and an angled intermediate third deodorant layer 37 that lies between and against the first and second angled layers 35 and 36. Therefore, the angled second deodorant layer 32 extends below the angled intermediate third deodorant layer 37, and the angled intermediate third deodorant layer 37 extends below the angled first deodorant layer 35. Initially, the first, second and third deodorant layers 35-37 lie adjacent one another at the top of the cylindrical deodorant applicator stick 30 at which to be simultaneously accessible to the user. In this same regard, the volume of the intermediate third deodorant layer 37 is greater than the volume of the first deodorant layer 35 but less than the volume of the second deodorant layer 36.

In each of the cylindrical multi-layer deodorant applicator sticks 28 and 30 of FIGS. 7 and 8, as the tops of the sticks wear down during use, the respective uppermost first deodorant layers 32 and 35 thereof will eventually be consumed so that the sensory characteristic provided thereby will no longer be available to the user. In each of these cases, only the deodorant composition layers (i.e., designated 33 for the deodorant applicator stick 28 and designated 36 and 37 for the deodorant applicator stick 30) that lie below the uppermost first layers will then be accessible to provide their sensory characteristics to the user. In other words, the potency of the angled uppermost first layers of the deodorant applicator sticks 28 and 30 is reduced over time.

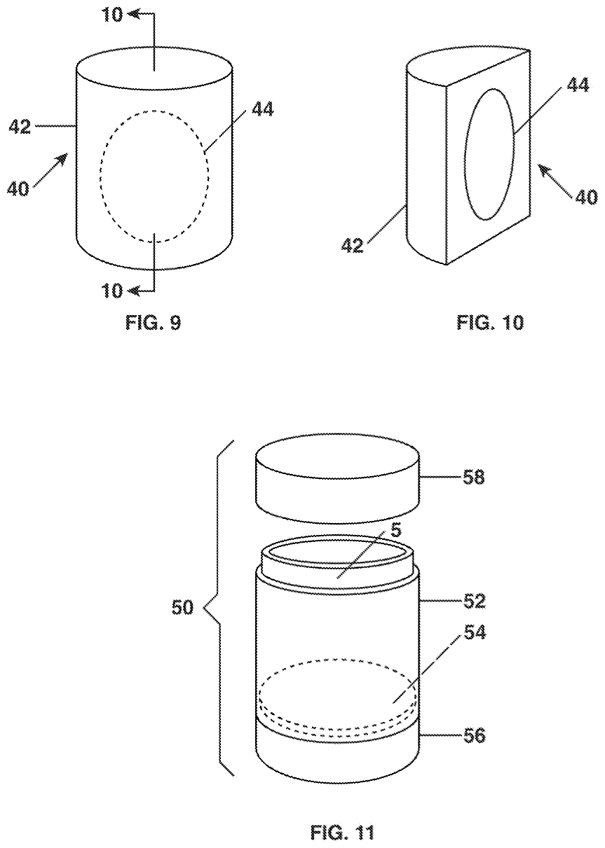

FIGS. 9 and 10 of the drawings show a ninth multi-layer deodorant applicator stick 40 including a cylindrical configuration with an upstanding cylindrical first deodorant composition layer 42 haying a first sensory characteristic and a second deodorant composition layer 44 having a different second sensory characteristic and extending radially through the cylindrical first layer 42. By way of example, the radially extending second layer 44 has a circular cross-section and is embedded within and runs in a widthwise direction through the upstanding cylindrical first layer 42. That is, the radially extending second layer 44 lies below the top, above the bottom, and within the sides of the cylindrical first layer 42 of the multi-layer deodorant applicator stick 40.

In this case, as the deodorant applicator stick 40 wears down during use, only the top of the cylindrical first deodorant composition layer 42 will be consumed so that the sensory characteristic thereof is solely available to the user. As the deodorant applicator stick 40 continues to wear down, the cylindrical first and the radially extending second deodorant composition layers 42 and 44 will be simultaneously consumed so that the sensory characteristics of both will be available to the user. However, because of the circular cross-section of the second layer 44 that extends radially through the upstanding cylindrical first layer. 42, the potency of the sensory characteristics provided by the first and second layers 42 and 44 will vary over time as the deodorant applicator stick wears down.

FIG. 11 of the drawings illustrates one example of a container 50 within which any one of the earlier disclosed multi-layer deodorant applicator sticks 1, 3, 13, 15, 24, 26, 28, 30 and 40 of FIGS. 1-10 may be carried and dispensed during use. However, it is to be understood that a container is optional and, therefore, is not to be considered a part or a limitation of this invention. The container 50 of FIG. 11 includes a cylindrical body 52 that is sized to receive one of the aforementioned cylindrical deodorant applicator sticks. An elevator platform 54 lies at the bottom of the body 52 on which a multi-layered deodorant applicator stick will be seated. A rotatable base 56 having a screw drive is located at the bottom of the container 50 to communicate with the elevator platform 54. A rotation of the base 56 correspondingly causes the elevator platform 54 to be raised such that any multi-layer deodorant applicator stick that is seated on the platform will be lifted upwardly and outwardly from the body 52 of the container 50 as the deodorant applicator stick is consumed during use. A screw-on lid 58 is removably attached to the top of the container 50 by which to close the container and seal the deodorant applicator stick therewithin for transport or storage.

By way of example, any of the multi-layer deodorant applicator sticks shown at FIGS. 1-10 of the drawings may be manufactured by pouring into a container (such as that designated 50 in FIG. 11) a liquid or a semi-solid first deodorant composition layer having a first sensory characteristic. After the first layer has congealed into a desired shape, a second deodorant composition layer having, a different sensory characteristic is poured into the container above or alongside the first layer. The second layer is then congealed into a desired shape adjacent the first layer. This step may be repeated any number of times.

Other methods may be used to manufacture the multi-layer deodorant applicator sticks herein disclosed. For example, two or more solid deodorant composition layers having different sensory characteristics may be heated and bonded one to the other. Likewise, a pressure can be applied to the two or more solid deodorant layers to attach the layers to one another. The shape and orientation of the layers in the container may be uniform or irregular in the manner described above. In this same regard, one of the layers may partially or completely surround and/or engage a second layer. Depending upon the shape and orientation of the layers, the user can have access to a customized mixture so as to be able to experience different sensory characteristics at the same time and selectively modulate his exposure to these characteristics over time from a single deodorant applicator stick.

By way of further example, each deodorant composition layer of the multi-layer deodorant applicator sticks herein disclosed can be produced by combining any number of ingredients that are necessary to provide the layer with its desired sensory characteristics, such as color and fragrance. The ingredients for the layer are preferably placed into a jacketed stainless steel kettle, or the like. Steam or any other suitable supply of heat is applied to melt any solid ingredients. The batch is blended in the kettle until the contents have a homogeneous composition. After being blended, the molten deodorant composition layer is poured via a filling nozzle into a container such as that described while referring to FIG. 11. The deodorant composition layer may be configured (e.g., molded) inside the container at slightly above its congealing temperature to any one of a variety of different shapes. The layer is then allowed to cool either naturally or by refrigeration. A second molten deodorant composition layer having a second sensory characteristic is then poured into the container to lie over and/or alongside the first layer to be configured in the same manner as just described.

Alternatively, two or more deodorant composition layers that have congealed and been shaped outside the container can be joined together by means of heat and/or pressure. The composite multi-layer deodorant applicator stick is then loaded into its container to be dispensed to the user therefrom.

According to a preferred embodiment, multi-layer deodorant applicator sticks which include different sensory characteristics have been shown and described above. However, it is to be understood that the teachings and advantages of this invention are also applicable to other solid and semi-solid (including gels) personal care applicators, the contents of which are applied to the user's body. By way of example, such personal care applicators to which this invention is applicable include lip balm, lipstick, sun block and a scented fragrance (e.g., perfume) applicator to name but a few.

1. A consumable multi-layer deodorant applicator stick to be applied to the body of a user, and comprising:
a first deodorant composition layer having a first sensory characteristic; and
at least one second deodorant composition layer having a different sensory characteristic,
said first and said at least one second deodorant composition layers being joined one to the other such that the application of said multi-layer deodorant applicator stick to the user's body provides the user with each of said first and said different sensory characteristics as said deodorant applicator stick is consumed during use.

2. The consumable multi-layer deodorant applicator stick recited in claim 1, wherein said first deodorant composition layer is positioned relative to said at least one second deodorant composition layer such that the user is provided with the first sensory characteristic prior to being provided with the different sensor characteristic.

3. The consumable multi-layer deodorant applicator stick recited in claim 1, wherein said first deodorant composition layer is positioned relative to said at least one second deodorant composition layer such that said first deodorant composition layer is consumed prior to the consumption of said at least one second deodorant composition layer.

4. The consumable multi-layer deodorant applicator stick recited in claim 1, wherein said first deodorant composition layer is positioned relative to said at least one second deodorant composition layer, such that said first and said at least one second deodorant composition layers are applied simultaneously to the user.

5. The consumable multi-layer deodorant applicator stick recited in claim 1, wherein the size of one of said first and said at least one second deodorant composition layers is greater than the size of the other one of said deodorant composition layers.

6. The consumable multi-layer deodorant applicator stick recited in claim 1, wherein said first deodorant composition layer and said at least one second deodorant composition layer have different shapes.

7. The consumable multi-layer deodorant applicator stick recited in claim 1, wherein said first deodorant composition layer lies partially above said at least one second deodorant composition layer.

8. The consumable multi-layer deodorant applicator stick recited in claim 1, wherein said first deodorant composition layer lies entirely above said at least one second deodorant composition layer.

9. The consumable multi-layer deodorant applicator stick recited in claim 8, wherein each of first deodorant composition layer and said at least one second deodorant composition layer has a disc shape, said first disc-shaped deodorant composition layer being seated on top of and lying above said at least one second disc-shaped deodorant composition layer.

10. The consumable multi-layer deodorant applicator stick recited in claim 1, wherein some of said at least one second deodorant composition layer lies below said first deodorant composition layer.

11. The consumable multi-layer deodorant applicator stick recited in claim 10, wherein each of said first deodorant composition layer and said at least one second deodorant composition layer has an angled face, said first deodorant composition layer laying against said at least one second deodorant composition layer such that the respective angled surfaces thereof lie face-to-face one another.

12. The consumable multi-layer deodorant applicator stick recited in claim 1, wherein said deodorant applicator stick has a cylindrical configuration, each said first deodorant composition layer and said at least one second deodorant composition layer also having a cylindrical configuration and being concentrically aligned one in surrounding engagement with the other.

13. The consumable multi-layer deodorant applicator stick recited in claim 1, wherein said deodorant applicator stick has a cylindrical configuration, and each of said first deodorant composition layer and said at least one second deodorant composition layer having a semi-cylindrical configuration and extending vertically and in parallel alignment with one another.

14. The consumable multi-layer deodorant applicator stick recited in claim 1, wherein said first deodorant composition layer is cylindrical, and said at least one second deodorant composition layer extends radially through said cylindrical first deodorant composition layer.

15. The consumable multi-layer deodorant applicator stick recited in claim 14, wherein said cylindrical first deodorant composition layer has a top and a bottom, and said at least one second deodorant composition layer that extends radially through said cylindrical first deodorant composition layer has a circular cross-section and lies between the top and the bottom of said cylindrical first deodorant composition layer.

16. A consumable multi-layer personal care applicator to be applied to the body of a user, and comprising:
a first layer having a first sensory characteristic; and
at least one second layer having a different sensory characteristic,
said first and said at least one second layers lying adjacent one another such that the application of said multi-layer personal care applicator to the user's body provides the user with each of said first and said different sensory characteristics as said personal care applicator is consumed during use.

17. The consumable multi-layer personal applicator recited in claim 16, wherein said layer is positioned relative to said at least one second layer such that the user is provided with the first sensory characteristic prior to being provided with the different sensory characteristic.

18. The consumable multi-layer personal applicator recited in claim 16, wherein said first layer is positioned relative to said at least one second layer such that said first layer is consumed prior to the consumption of said at least one second layer.

19. The consumable multi-layer personal applicator recited in claim 16, wherein said first layer is positioned relative to said at least one second layer, such that said first and said at least one second layers are applied simultaneously to the user.
